(19) United States
(12) Patent Application Publication
(10) Pub. No.: US 2012/0258945 A1
(43) Pub. Date: Oct. 11, 2012

(54) Association Between Ferroquine and an Artemisinine Derivative for Treating Malaria

(75) Inventors: Laurent FRAISSE, Balma (FR); Daniel TER-MINASSIAN, Fontenay aux Roses (FR)

(73) Assignee: SANOFI-AVENTIS, Paris (FR)

(21) Appl. No.: 11/874,377

(22) Filed: Oct. 18, 2007

Related U.S. Application Data
(63) Continuation of application No. PCT/FR2006/000842, filed on Apr. 18, 2006.

(30) Foreign Application Priority Data
Apr. 20, 2005 (FR) 0503932

(51) Int. Cl. A61K 31/555; A61J 1/00; A61P 17/00

(52) U.S. Cl. 514/187; 206/570

(57) ABSTRACT

This invention is directed to methods for the treatment and prevention of malaria comprising administering a combination of ferroquine, or a pharmaceutically acceptable salt, hydrate or solvate thereof, and an artemisinin derivative, and to pharmaceutical compositions comprising such combination.

The present invention relates to a novel combination of antimalarial active ingredients, i.e. ferroquine and an artemisinin derivative, and also to a pharmaceutical composition comprising such a combination, that is useful for the treatment and/or prevention of malaria.

### BACKGROUND OF THE INVENTION

Malaria is one of the primary infectious causes of mortality in the world and annually affects more than 500 million individuals, among whom 3 million die each year. This scourge affects mainly sub-Saharan Africa, South-East Asia and Latin America.

Four types of parasites of the Plasmodium genus (P. falciparum, P. malariae, P. vivax and P. ovale), transported by Anopheles mosquitoes, propagate malaria. Plasmodium falciparum, which is widespread in Africa, is the most virulent parasite and is responsible for the lethal forms of the disease.

The strong upsurge in the disease observed over the last few years is due to several factors, among which is the resistance of many strains of Plasmodium falciparum to the medicinal products conventionally used, such as chloroquine, mefloquine, amodiaquine or else pyrimethamine.

Isolated in 1972 from the plant Artemisia annua (qinghaosu), and used for centuries in China, artemisinin has a powerful antimalarial activity. Derivatives with enhanced pharmacological properties, such as artemether, arteether and artesunate, are also marketed.

Artemisinin and its derivatives are today among the most effective active ingredients against Plasmodium falciparum. However, the use of artemisinin or of its derivatives in monotherapy could be a causal factor in the selection of resistant parasitic strains.

The scientific community now recommends the use of combinations of active ingredients, and in particular of combinations of artemisinin or of its derivatives with other antimalarial active ingredients. These polytherapies, called ACT (Artemisinin-based Combination Therapies), have been recommended by the World Health Organization (WHO) since 2002. They offer multiple advantages: improvement of therapeutic efficacy on resistant strains, protection of the two active ingredients against the appearance of resistance, reduction in disease transmission and in resistance propagation.

The combination of artemether and lumefantrine, marketed under the name Coartem®, has, for example, been proposed, as has the combination of artesunate and amodiaquine (Arsucam®).

In accordance with the strategy supported by the WHO, the search for novel combinations of antimalarial active ingredients must be continued.

Among the various antimalarial active ingredients described in the literature, ferroquine is a molecule that is active against chloroquine-resistant strains of Plasmodium falciparum. Ferroquine, also called ferrocene-chloroquine or ferrochloroquine, corresponds to 7-chloro-4-[({2-[(N,N-dimethyl-amino)methyl]ferrocenyl}methyl)amino]quinoline. It is a derivative of 4-aminoquinoline coupled to a ferrocene ring. This molecule is described in particular in patent EP 0 824 536 and in J. Med. Chem., 1997, 40, 3715-3718, Antimicrob. Agents Chemother., 1998, 42, 540-544, J. Org. Chem., 1999, 589, 59-65 and J. Organometallic Chem., 2004, 689, 4678-4682.

### SUMMARY OF THE INVENTION

Although the combination of chloroquine and artesunate does not achieve satisfactory levels of efficacy (Am. J. Trop. Med. Hyg., 2003, 69(1), 19-25 and Transactions of the Royal Society of Tropical Medicine and Hygiene, 2003, 97, 429-433) and can induce the appearance of resistant strains, in particular chloroquine-resistant strains, it has now been found, surprisingly, that a combination of ferroquine and an artemisinin derivative, in particular artesunate, artemether or arteether, is effective for the treatment and/or prevention of malaria, and in particular for anticipating and reducing, or even avoiding, the development of parasitic strains resistant to the two active ingredients, when the latter are administered as a monotherapy.

### BRIEF DESCRIPTION OF THE DRAWING

FIG. 1 shows the percentage survival of the animals from the fifth day after infection.

### DETAILED DESCRIPTION OF THE INVENTION

A subject of the present invention is therefore a novel combination of ferroquine (molecule (I) represented below in free base form and where Fe represents a ferrocene ring) and an artemisinin derivative.

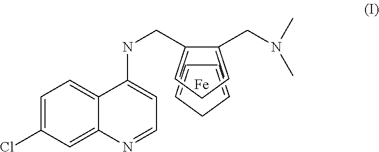

In the combinations according to the invention, the ferroquine may be in the form of a free base, but also in the form of a salt, of a hydrate or of a solvate (the latter being defined as associations or combinations of ferroquine with, respectively, one or more molecules of water or a solvent. The ferroquine is advantageously used in free base form.

The artemisinin derivative present in the combinations according to the invention advantageously consists of artesunate (II) or artemether (III):

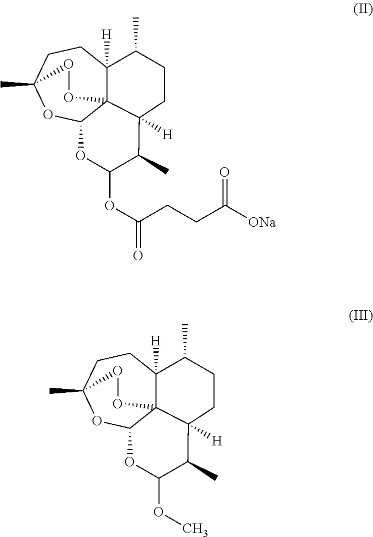

The subject of the invention is also a pharmaceutical composition comprising, as active ingredients, a combination of ferroquine (I) and an artemisinin derivative, advantageously artesunate (II) or artemether (III).

Such a pharmaceutical composition contains therapeutically effective doses of ferroquine, or of a pharmaceutically acceptable salt, of a hydrate or of a solvate of ferroquine, and of at least one artemisinin derivative, and also at least one pharmaceutically acceptable excipient. Said excipients are chosen, according to the pharmaceutical form and the method of administration desired, from the usual excipients which are known to those skilled in the art.

The suitable unit administration forms comprise forms for oral administration, such as tablets, soft or hard gelatine capsules, powders, granules and oral solutions or suspensions, forms for sublingual, buccal, intratracheal, intraocular or intranasal administration, or for administration by inhalation, forms for topical, transdermal, subcutaneous, intramuscular or intravenous administration, forms for rectal administration, and implants. For topical application, the compounds according to the invention can be used in creams, gels, ointments or lotions.

Preferred routes of administration are oral administration, rectal administration and injectable administration.

For example, when a solid composition in the form of tablets is prepared, the active ingredients are mixed with one or more pharmaceutical excipients, such as gelatine, starch, lactose, magnesium stearate, talc, silica, gum arabic, mannitol, microcrystalline cellulose, hydroxypropylmethylcellulose, croscarmellose or the like. The tablets can be coated with sucrose, with a cellulose derivative or with other materials suitable for coating. The tablets can be produced by various techniques, such as direct compression, dry granulation, wet granulation or hot melt.

It is also possible to obtain a preparation in the form of gelatine capsules by mixing the active ingredients with a diluent and pouring the mixture obtained into soft or hard gelatine capsules.

For parenteral administration, use is made of aqueous suspensions, isotonic saline solutions or sterile and injectable solutions which contain dispersing agents and/or wetting agents that are pharmacologically compatible, for example propylene glycol or butylene glycol.

For oral administration, the daily doses of each of the two active ingredients of the combination according to the invention are as follows:
ferroquine: between 50 and 1600 mg, preferably between 200 and 1200 mg, even more preferably between 400 and 800 mg per individual and per day;
artemisinin derivative: between 1 and 10 mg/kg/day, preferably between 2 and 6 mg/kg/day, even more preferably approximately 4 mg/kg/day.

There may be specific cases where higher or lower dosages are appropriate; such dosages do not depart from the context of the invention. According to the usual practice, the dosage appropriate for each patient is determined by the physician according to the method of administration and the weight and response of said patient.

The combination according to the invention is intended to be administered for 3 consecutive days, taken as one or more daily doses of each of the two active ingredients, preferably taken as a single dose per day. This treatment time limited to 3 days is particularly advantageous, in comparison with the 7 days recommended for a monotherapy with the artemisinin derivatives, in that it allows better adherence to the treatment by the patients, thus avoiding the premature interruptions of the treatment which, in the long term, induce resistance of the parasite.

The administration of each of the two active ingredients can be carried out simultaneously, or else separately or spread out over time (sequential administration).

When the administration is carried out simultaneously, the two active ingredients can be combined within a single pharmaceutical form (fixed combination), such as a tablet or a gelatine capsule suitable for oral administration.

The two active ingredients of the combination according to the invention can also, regardless of whether or not their administration is simultaneous, be present in distinct pharmaceutical forms. To this effect, the combinations according to the invention can be in the form of a kit comprising, firstly, ferroquine or a salt, hydrate or solvate of ferroquine and, secondly, at least one artemisinin derivative such as artesunate or artemether, said ferroquine and said artemisinin derivative being in distinct compartments and being intended to be administered simultaneously, separately or spread out over time (sequential administration).

By way of example, a unit administration form of ferroquine in the form of a tablet can comprise the following components:

| | |
|---|---|
| Ferroquine | 50 mg |
| Mannitol | 224 mg |
| Sodium croscaramellose | 6 mg |
| Corn starch | 15 mg |
| Hydroxypropylmethylcellulose | 2 mg |
| Magnesium stearate | 3 mg |

Also by way of example, a unit administration form of artesunate in the form of a tablet can comprise 50 or 100 mg of artesunate and usual excipients, for example lactose, croscarmellose, anhydrous colloidal silica, microcrystalline cellulose and magnesium stearate.

A subject of the present invention is also a method of treatment and/or of prevention of malaria, which comprises the administration, to a patient, of a therapeutically effective dose of ferroquine, or of a pharmaceutically acceptable salt, of a hydrate or of a solvate of ferroquine, and of a therapeutically effective dose of at least one artemisinin derivative, said doses being administered simultaneously or else sequentially to said patient, as is described above.

The combination according to the invention has been the subject of in vivo biochemical tests in mice infected with a plasmodium of Plasmodium falciparum type (strain Plasmodium vinckei vinckei), making it possible to demonstrate its effectiveness for the treatment of malaria.

The tests below, carried out with artesunate, are given by way of example. Since the artemisinin derivatives all have the same metabolite (dihydroartemisinin) and a short half-life in common, the results of these tests, obtained for artesunate, can be generalized to the other artemisinin derivatives, such as artemether or arteether.
Measurement of the In Vivo Activity in Mice Infected with Plasmodium Vinckei of Ferroquine, of Artesunate and of the Combination of the Two Compounds

1. Description of the In Vivo Test Used

Female “Swiss” mice, aged eight weeks and one day, are inoculated with parasites of Plasmodium vinckei vinckei type (Rodhain, 1952). The mice are acclimatized beforehand for two weeks. The mice are given food and drink ad libitum.

The Plasmodium vinckei vinckei strain is maintained by weekly infection in the mouse with 10⁷ parasitized erythrocytes suspended in a phosphate buffered saline (0.9%).

On the first day of treatment (D0), one hour after infection (10⁷ parasitized erythrocytes suspended in a phosphate buffered saline (0.9%)), the animal is administered, orally, as appropriate, with ferroquine, with artesunate or with a mixture of the two active ingredients. This administration is repeated on the following three days (D1 to D3) (Peter, 1987). When the two products are administered in combination, the artesunate is administered first, the ferroquine is administered second, 45 minutes later. On the fourth day, a blood smear is taken from the tail of the mouse. The sample is fixed on a plate. The number of parasitized blood cells is counted under a microscope. The parasitaemia is expressed as percentage of infected erythrocytes present in the sample on a sample of 1000 cells. Six or seven mice are used per dose.

The mice for which the smear at D4 shows no trace of parasites will again be checked on the 10th, 17th, 24th, 31st, 38th, 45th, 52nd and 59th day in order to detect any possible upsurge of parasites.

### Preparation of the Suspensions of Dilutions and for Administration of the Compounds

Preparation of the Ferroquine Suspension

(ferroquine from Sanofi-Synthélabo, batch MY18.0088)

Ferroquine is mixed with methylcellulose (0/5 (w/w)) and Polysorbate 80 (0/5 (w/w)). The preparation is stable for at least 7 days in the dark, in the cold (4° C.), and for 4 hours at ambient temperature. The final suspension of ferroquine has a concentration ranging between 0.1 and 100 mg/ml.

Preparation of the Artesunate Suspension

(artesunate from Sanofi-Synthelabo, batch 1.04)
Artesunate is mixed with methylcellulose (0/5 (w/w)) and Polysorbate 80 (0/5 (w/w)). The preparation is stable for 4 hours in the dark and at ambient temperature. The final suspension of artesunate has a concentration ranging between 0.8 and 20 mg/ml.

2. Determination of the IC₅₀ Values and of the Curative Doses of Artesunate and of Ferroquine Administered Separately

### Method of Determination of the IC₅₀ Values

The IC₅₀ is defined as the concentration in mg/kg/day which inhibits the blood parasitaemia by 50% at the fourth day (D4) after infection (D0) and four days of treatment (D0,D1,D2,D3). 0% inhibition corresponds to the mean of the parasitaemias observed in the untreated infected mice. 100% inhibition corresponds to a very low or zero parasitaemia, less than 0.1%. The IC₅₀ values are determined by linear interpolation of the dose-response curve represented as logarithm of concentrations.

The IC₅₀ of ferroquine is determined after administration of concentrations of between 1 and 10 mg/kg/day. The concentrations used are 0, 1, 1.47, 2.1, 3.2, 4.6, 6.8 and 10 mg/kg/day for 4 days.
The IC₅₀ of artesunate is determined after administration of concentrations of between 1 and 15 mg/kg/day. The concentrations used are 0, 1, 1.6, 2.5, 3.9, 6.1, 9.5 and 15 mg/kg/day for 4 days.
The IC₅₀ values obtained are given in Table I below:

TABLE I

| | IC₅₀ (mg/kg/day) |
|---|---|
| ferroquine | 3.32 |
| artesunate | 2.79 |

For ferroquine, the curative dose is 10 mg/kg/day.
For artesunate, the curative dose was not reached in this study and is therefore greater than 15 mg/kg/day.

It is important to carry out the interaction study with non-curative (sub-optimal) doses of artesunate and ferroquine, which are therefore close to the IC₅₀ values obtained with the two compounds studied separately.

Ferroquine has a curative dose (complete survival of the mice treated) close to the IC₅₀ value. For artesunate, the difference between the curative dose and the IC₅₀ value is greater and a dose greater than the IC₅₀ value can therefore be used. Joint and separate administrations of 3 mg/kg/day for ferroquine and 6 mg/kg/day for artesunate, for 4 days, were therefore considered during the combination study.

3. Measurement of the Antimalarial Activity of the Ferroquine/Artesunate Combination on a Strain of Plasmodium Vinckei Vinckei Parasite In Vivo

3.1. Determination of the Percentage of Parasitaemia

Combined and separate administrations of 3 mg/kg/day for ferroquine and 6 mg/kg/day for artesunate, for 4 days, were carried out on infected mice, in comparison with a batch that did not receive treatment. Table II indicates the mean parasitaemias (percentage of infected erythrocytes) observed at the fourth day after infection.

TABLE II

| | |
|---|---|
| Parasitaemia (as | Artesunate |
| %) | (mg/kg/day) |

| ferroquine | 0 | 6 |
|---|---|---|
| (mg/kg/day) | mean | mean |
| 0 | 31.4 | 7.1 |
| 3 | 8.8 | 0.16 |

As indicated in Table II, the combined administration of ferroquine at the dose of 3 mg/kg/day and of artesunate at the dose of 6 mg/kg/day, for 4 days, makes it possible to significantly reduce the parasitaemia of the infected animals compared with the separate administration of the two products.

3.2. Determination of the Mortality of the Mice

Each day, from day 5 after infection, the number of dead mice is counted so as to determine the percentage mortality (number of mice dead relative to the number of mice of the batch considered). The curative dose is the first dose at which all the mice of the batch survive.

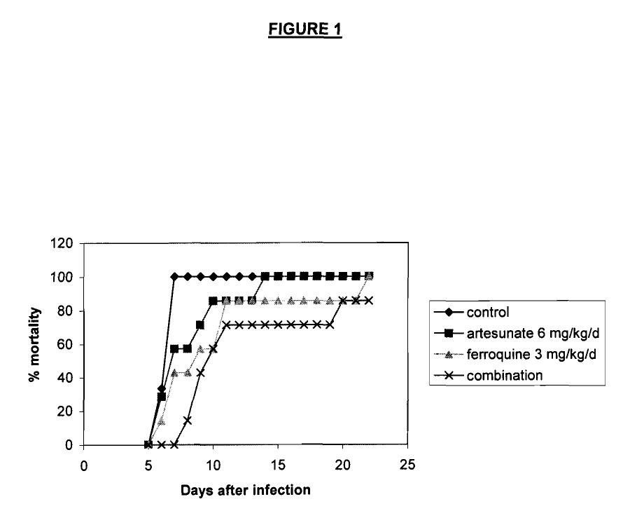

FIG. 1 shows the percentage survival of the animals from the fifth day after infection.

As shown in FIG. 1, the period of survival of the animals is improved by the combined administration of sub-optimal doses of compounds (ferroquine at the dose of 3 mg/kg/day and artesunate at the dose of 6 mg/kg/day for 4 days) compared with the separate administrations (ferroquine at the dose of 3 mg/kg/day or artesunate at the dose of 6 mg/kg/day for 4 days).

The results obtained in vivo in mice infected with P. vinckei vinckei clearly demonstrate the absence of antagonism between the two active ingredients, and prove that the combination, according to the invention, of artesunate (or of an artemisinin derivative in general) and of ferroquine is advantageous for the treatment of malaria.

1. A method for the treatment of malaria in a patient in need thereof, comprising administering to such patient a therapeutically effective amount of a combination of ferroquine, in the form of a free base or a pharmaceutically acceptable salt thereof, and an artemisinin derivative.

2. A method according to claim 1, wherein the artemisinin derivative is selected from group consisting of artesunate, arteether and artemether.

3. A method according to claim 2, wherein the artemisinin derivative is selected from the group consisting of artesunate and artemether.

4. A method according to claim 1 wherein the ferroquine, or pharmaceutically acceptable salt thereof, is administered in a dose of between 50 mg and 1600 mg per person per day.

5. A method according to claim 4 wherein the dose administered is between 200 mg and 1200 mg per person per day.

6. A method according to claim 5 wherein the dose administered is between 400 mg and 800 mg per person per day.

7. A method according to claim 1 wherein the artemisinin derivative is administered in a dose of between 1 mg/kg and 10 mg/kg per day.

8. A method according to claim 7 wherein the dose administered is between 2 mg/kg and 6 mg/kg per day.

9. A method according to claim 8 wherein the dose administered is about 4 mg/kg per day.

10. A method according to claim 1 wherein the ferroquine, or pharmaceutically acceptable salt thereof, is administered in a dose of between 50 mg and 1600 mg per person per day, and wherein the artemisinin derivative is administered in a dose of between 1 mg/kg and 10 mg/kg per day.

11. A method according to claim 1 wherein the ferroquine, or pharmaceutically acceptable salt thereof, and the artemisinin derivative are administered over a period of 2, 3 or 4 consecutive days.

12. A method according to claim 1 wherein the ferroquine, or pharmaceutically acceptable salt thereof, and the artemisinin derivative are administered simultaneously.

13. A method according to claim 1 wherein the ferroquine, or pharmaceutically acceptable salt thereof, and the artemisinin derivative are administered sequentially.

14. A pharmaceutical composition comprising a therapeutically effective dose of ferroquine, or a pharmaceutically acceptable salt thereof, and a therapeutically effective amount of at least one artemisinin derivative, and at least one pharmaceutically acceptable excipient.

15. A pharmaceutical composition comprising a therapeutically effective dose of a combination of ferroquine, or a pharmaceutically acceptable salt thereof, and at least one artemisinin derivative, and at least one pharmaceutically acceptable excipient.

16. A pharmaceutical composition according to claim 14 wherein the artemisinin derivative is selected from the list consisting of artesunate, arteether and artemether.

17. A pharmaceutical composition according to claim 16 wherein the artemisinin derivative is selected from the list consisting of artesunate and artemether.

18. A pharmaceutical composition according to claim 15 wherein the artemisinin derivative is selected from the list consisting of artesunate, arteether and artemether.

19. A pharmaceutical composition according to claim 18 wherein the artemisinin derivative is selected from the list consisting of artesunate and artemether.

20. A method according to claim 1 wherein the ferroquine, or pharmaceutically acceptable salt thereof, is administered orally, rectally or by infection, and the artemisinin derivative is administered orally, rectally or by injection.

21. A kit for the treatment or of malaria, comprising, firstly, ferroquine or a pharmaceutically acceptable salt thereof and, secondly, at least one artemisinin derivative, said ferroquine and said artemisinin derivative being in distinct compartments and being intended to be administered simultaneously or sequentially.
